(19) United States
(12) Patent Application Publication
(10) Pub. No.: US 2022/0194962 A1
(43) Pub. Date: Jun. 23, 2022

(54) AZAZINOINDAZOLE DERIVATIVE, PREPARATION METHOD THEREFOR AND USE THEREOF

(71) Applicant: CHENGDU SCIMOUNT PHARMATECH CO., LTD., Chengdu, Sichuan (CN)

(72) Inventors: Dejian XIE, Chengdu, Sichuan (CN); Jinkun HUANG, Chengdu, Sichuan (CN); Chunchao YUE, Chengdu, Sichuan (CN); Wei ZHANG, Chengdu, Sichuan (CN); Fang CHEN, Chengdu, Sichuan (CN); Xiaoxia LI, Chengdu, Sichuan (CN)

(21) Appl. No.: 17/594,568

(22) PCT Filed: Apr. 21, 2020
(86) PCT No.: PCT/CN2020/085775

§ 371 Date: Mar. 14, 2022

(30) Foreign Application Priority Data
Apr. 22, 2019 (CN) 201910325129.2

(51) Int. Cl. C07D 519/00; A61K 45/06

(52) U.S. Cl. CPC C07D 519/00; A61K 45/06

(57) ABSTRACT

An azazinoindazole derivative represented by formula I, a pharmaceutically acceptable salt thereof, a stereoisomer thereof, a tautomer thereof, a prodrug thereof, a solvate thereof or a hydrate thereof are presented. The described compound may be used as a 5-HT3 receptor modulator. Experiments have proven that the compound of the preparation example has a strong affinity to 5-HT3 receptors, and has high activity as a modulator of 5-HT3 receptors. The compound may be used to prepare a drug for treating irritable bowel syndrome, nausea, vomiting, gastroenteritis, gastric dysfunction, diarrhea, pain, carcinoid syndrome, drug addiction and other diseases.

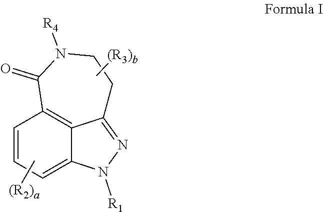

### TECHNOLOGY FILED

The present invention belongs to the field of pharmaceutical chemistry, and in particular relates to an azepinoindazole derivative, as well as the preparation method thereof and the use thereof.

### BACKGROUND TECHNOLOGY

The receptor of 5-hydroxytryptamine type 3 (also known as serotonin type 3, 5-HT₃) is a part of serotonin system. It is known that 5-HT₃ receptor is expressed in the central nervous system involving vomiting reflex, pain processing, cognition and anxiety control, and plays a regulatory role in the pathogenesis of nausea, vomiting, migraine, drug addiction, and neurodegenerative and psychiatric disorders. In addition, 5-HT3 receptor is also expressed in the gastrointestinal tract, which can regulate gastrointestinal diseases such as dyspepsia, gastroesophageal reflux disease and irritable bowel syndrome.

Irritable bowel syndrome (IBS) is a continuous or intermittent gastrointestinal disorder. Its clinical manifestations are abdominal pain, abdominal distension, changes in defecation habits and/or stool characteristics, lack of gastrointestinal structure and biochemical abnormalities. IBS is one of the most common gastrointestinal diseases in clinic, whose patients are mainly young and middle-aged people, with a common onset age of 20 to 50 years old. Female patients are more common than male patients, and patients have a tendency of family aggregation. IBS is often accompanied by other gastrointestinal disorders such as functional dyspepsia. According to the characteristics of stool, IBS is divided into four clinical types: diarrhea type, constipation type, mixed type and uncertain type, of which diarrhea is the main type.

Carcinoid is a rare slow-growing tumor that can produce small molecular peptides or peptide hormones, and as the most common endocrine tumor in the gastrointestinal tract, it has been classified as neuroendocrine neoplasms (NENS). Carcinoid syndrome is mainly treated by somatostatin analogues (SSAs) to control symptoms. However, for adult patients with carcinoid syndrome diarrhea (CSD) whose condition cannot be fully controlled by SSAs therapy alone, targeting tryptophan hydroxylase (TPH) in carcinoid tumor cells and inhibiting the excessive production of serotonin can reduce the frequency of carcinoid syndrome diarrhea and significantly alleviate the symptoms.

The average incidence of chemotherapy-induced nausea and vomiting (CINV) is as high as 90%. CINV may make the patient's resistance worse and worse, delay the improvement of the disease, and cause negative results to the patient.

Postoperative nausea and vomiting (PONV) are the most common symptom after operation. Affected by many factors such as operation type, operation duration, anesthetic drugs and methods, preoperative anxiety and so on, most patients will have PONV 24 hours after operation, and obvious nausea symptoms will appear before vomiting. Postoperative nausea and vomiting are also a common postoperative complication. It is reported that the average incidence is 20% to 30%, and even 70% in high-risk patients. In addition to bringing severe discomfort to patients, nausea and vomiting may also cause medical complications, such as wound dehiscence, bleeding, aspiration pneumonia, water-electrolyte disorder, etc. Therefore, the prevention and treatment of PONV has attracted more and more attention.

Studies have shown that regulating 5-HT₃ receptor has therapeutic effects on IBS, CSD, CINV and PONV. At present, the drug approved for the treatment of IBS by regulating the activity of 5-HT₃ receptor is 5-HT₃ receptor antagonist, which is a drug that can completely antagonize the activity of 5-HT₃ receptor, but the drugs in the prior art can not fully meet the clinical needs. Therefore, the preparation of more effective and safer 5-HT₃ receptor modulators that can meet clinical needs is the key to the treatment of a variety of diseases including IBS, CSD, CINV and PONV.

### CONTENT OF THE INVENTION

The object of the present invention is to provide a more effective and safer 5-HT₃ receptor regulator.

The present invention provides a compound of formula I, a pharmaceutically acceptable salt thereof, a stereoisomer thereof, a tautomer thereof, a prodrug thereof, a solvate thereof or a hydrate thereof:

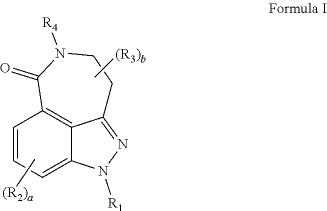

wherein, each of R₁, R₂, R₃, and R₄ is independently selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, substituted or unsubstituted alkenyl, substituted or unsubstituted alkynyl, substituted or unsubstituted alkyl, substituted or unsubstituted alkoxy, substituted or unsubstituted monoheterocyclyl, substituted or unsubstituted monocycloalkyl, substituted or unsubstituted fused azacycloalkyl, substituted or unsubstituted bridged azacycloalkyl, substituted or unsubstituted azabi(cycloalkyl), substituted or unsubstituted azaspirocycloalkyl, substituted or unsubstituted fused cycloalkyl, substituted or unsubstituted bridged cycloalkyl, substituted or unsubstituted bi(cycloalkyl), and substituted or unsubstituted spirocycloalkyl;
above substitutents are each independently selected from the group consisting of deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, C₂₋₈ alkenyl, C₂₋₈ alkynyl, C₁₋₈ alkyl, and C₁₋₈ alkoxy;
wherein, each of L₀, L₁, L₂, L₃, L₄, L₅, and L₆ is independently selected from 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, and C₁₋₈ alkyl;
a is selected from an integer of 1 to 3, and b is selected from an integer of 1 to 4.

Further,

R₂ is selected from the group consisting of deuterium, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, substituted or unsubstituted C₂₋₈ alkenyl, substituted or unsubstituted C₂₋₈ alkynyl, substituted or unsubstituted C₁₋₈ alkyl, substituted or unsubstituted C₁₋₈ alkoxy, substituted or unsubstituted 3-10 membered monoheterocyclyl, substituted or unsubstituted 3-10 membered monocycloalkyl, substituted or unsubstituted 3-10 membered fused azacycloalkyl, substituted or unsubstituted 3-10 membered bridged azacycloalkyl, substituted or unsubstituted 3-10 membered azabi(cycloalkyl), substituted or unsubstituted 3-10 membered azaspirocycloalkyl, substituted or unsubstituted 3-10 membered fused cycloalkyl, substituted or unsubstituted 3-10 membered bridged cycloalkyl, substituted or unsubstituted 3-10 membered bi(cycloalkyl), and substituted or unsubstituted 3-10 membered spirocycloalkyl;
above substituents, L₀, L₁, L₂, L₃, L₄, L₅, L₆, R⁶, R⁷, R⁸, R⁹, R¹⁰, and R¹¹ are as described above;
R₁, R₃, R₄, a, and b are as described above;

Further, said compound has a structure of formula II-1a:

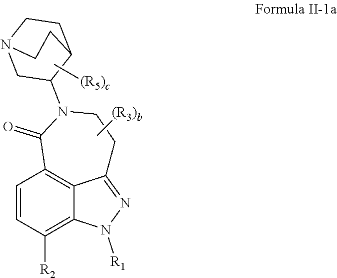

wherein, R₂ is selected from the group consisting of deuterium, cyano or hydroxyl;
R₅ is selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, C₂₋₈ alkenyl, C₂₋₈ alkynyl, C₁₋₈ alkyl, C₁₋₈ alkoxy; wherein, each of L₀, L₁, L₂, L₃, L₄, L₅, and L₆ is independently selected from the group consisting of 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, C₁₋₈ alkyl;
c is selected from an integer of 1 to 5;
R₁, R₃, and b are as described above.

Further, said compound has a structure of formula II-2a:

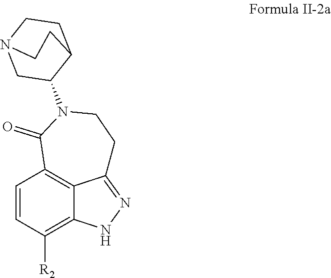

wherein, R₂ is selected from the group consisting of deuterium, cyano or hydroxyl.

Further, R₂ is selected from the group consisting of hydrogen or halogen;
R₁ is selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, substituted or unsubstituted C₂₋₈ alkenyl, substituted or unsubstituted C₂₋₈ alkynyl, substituted or unsubstituted C₁₋₈ alkoxy, substituted or unsubstituted 3-10 membered saturated monoheterocyclyl, substituted or unsubstituted 3-10 membered saturated monocycloalkyl, substituted or unsubstituted 3-10 membered saturated fused azacycloalkyl, substituted or unsubstituted 3-10 membered saturated bridged azacycloalkyl, substituted or unsubstituted 3-10 membered saturated azabi(cycloalkyl), substituted or unsubstituted 3-10 membered saturated azaspirocycloalkyl, substituted or unsubstituted 3-10 membered saturated fused cycloalkyl, substituted or unsubstituted 3-10 membered saturated bridged cycloalkyl, substituted or unsubstituted 3-10 membered saturated bi(cycloalkyl), and substituted or unsubstituted 3-10 membered saturated spirocycloalkyl;
   each of above substituents is independently selected from the group consisting of deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, C₂₋₈ alkenyl, C₂₋₈ alkynyl, C₁₋₈ alkyl, and C₁₋₈ alkoxy;
   wherein, each of L₀, L₁, L₂, L₃, L₄, L₅, and L₆ is independently selected from 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, and C₁₋₈ alkyl;
   a, b, R₃, and R₄ are as described above.

Further, said compound has a structure of formula III-1:

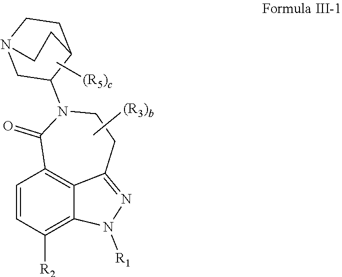

wherein, R₂ is selected from the group consisting of hydrogen or halogen;
   R₅ is selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, C₂₋₈ alkenyl, C₂₋₈ alkynyl, C₁₋₈ alkyl, C₁₋₈ alkoxy; wherein, each of L₀, L₁, L₂, L₃, L₄, L₅, and L₆ is independently selected from 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, C₁₋₈ alkyl;
c is selected from an integer of 1 to 5;
R₁, R₃, and b are as described above.

Further, said compound has a structure of formula III-2:

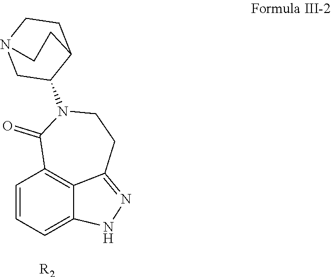

Wherein, R₂ is selected from halogen, and preferably fluorine, chlorine or bromine.

Further, said pharmaceutically acceptable salts include hydrochloride, hydrobromate, sulfate, phosphate, methanesulfonate, trifluoromethanesulfonate, benzenesulfonate, p-toluenesulfonate, 1-naphthalenesulfonate, 2-naphthalenesulfonate, acetate, trifluoroacetate, malate, tartrate, citrate, lactate, oxalate, succinate, fumarate, maleate, benzoate salicylate, phenylacetate, and mandelate. The present invention also provides a preparation method of the compound of above formula II-2a or III-2, characterized in that compound 9a has a deprotection reaction with a deprotection agent, to remove the group -PMB and obtain the final compound; the structure of compound 9a is

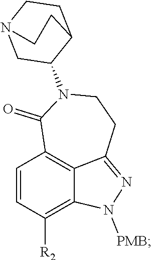

or, compound 9 is first subjected to a substitution reaction with a substitution reagent, to substitute Br in compound 9 with R₂ (other than Br) and obtain an intermediate product; then, the intermediate product has a deprotection reaction with the deprotection agent to remove the group -PMB and obtain the final compound; the structure of compound 9 is

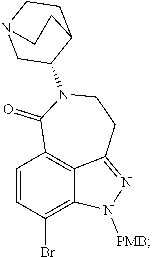

said final compound is the compound of above formula II-2a or 111-2;
said R₂ is selected from the group consisting of deuterium, cyano, hydroxyl or halogen, and said halogen is preferably fluorine, chlorine or bromine;
said deprotection agent is preferably trifluoroacetic acid; said substitution reagent is preferably zinc cyanide, 8-hydroxyquinaldine, deuterium gas, N-fluorobenzenesulfonimide or tetramethylammonium chloride.

In compound 9a, when R₂ is Br, it is compound 9.

Further, the preparation method of compound 9a comprises the following steps:

(1) Compound 1b reacts with compound 1a, to obtain compound 2b;
(2) Compound 2b reacts with sodium nitrite, to obtain compound 3b;
(3) Compound 3b reacts with p-methoxybenzyl bromide, to obtain compound 4b;
(4) Compound 4b reacts with (methoxymethyl)triphenylphosphonium chloride, to obtain compound 5b;
(5) Compound 5b reacts with hydrochloric acid, to obtain compound 6b;
(6) Compound 6b reacts with compound 7, to obtain compound 8b;
(7) Compound 8b reacts with K₂CO₃, to obtain compound 9a;
wherein, the structure of compound 1b is

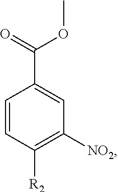

the structure of compound 1a is

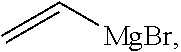

the structure of compound 2b is

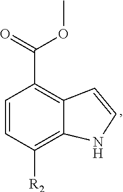

the structure of compound 3b is

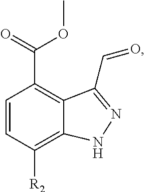

the structure of compound 4b is

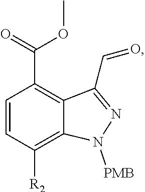

the structure of compound 5b is

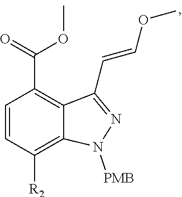

the structure of compound 6b is

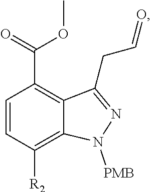

the structure of compound 7 is

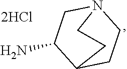

the structure of compound 8b is

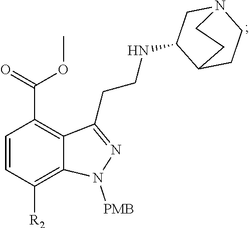

R₂ is as described above.

The present invention also provides the use of the compound mentioned above, a pharmaceutically acceptable salt thereof, a stereoisomer thereof, a tautomer thereof, a prodrug thereof, a solvate thereof or a hydrate thereof in the preparation of 5-HT3 receptor modulator.

Further, said 5-HT3 receptor modulator is 5-HT3 receptor antagonist or 5-HT3 receptor partial agonist.

Further, said 5-HT3 receptor modulator is a drug for treating diseases, and said diseases include generalized anxiety disorder, social phobia, vertigo, obsessive-compulsive disorder, panic disorder, post-traumatic stress disorder, bulimia nervosa, abstinence reaction, alcohol dependence, pain, sleep related central apnea, chronic fatigue syndrome, central nervous system related diseases, psychosis associated with Parkinson's disease, schizophrenia, cognitive impairment and deficits in schizophrenia, Parkinson's disease, Huntington's disease, presenile dementia, Alzheimer's disease, obesity, drug abuse disorders, dementia associated with neurodegenerative diseases, cognitive impairment, fibromyalgia syndrome, acne erythematosa, 5-hydroxytryptamine-mediated cardiovascular diseases, nausea, vomiting, gastrointestinal diseases, gastroesophageal reflux disease, Burkitt lymphoma, bronchial asthma, pruritus, migraine, epilepsy, carcinoid syndrome and irritable bowel syndrome; said nausea or vomiting is preferably that caused by chemotherapy, surgery, and radiotherapy.

The present invention also provides a pharmaceutical composition, which is a preparation obtained by using the compound mentioned above, a pharmaceutically acceptable salt thereof, a stereoisomer thereof, a tautomer thereof, a prodrug thereof, a solvate thereof or a hydrate thereof as the active ingredient, with the addition of pharmaceutically acceptable excipients.

The present invention also provides a combined drug, which contains the compound mentioned above, a pharmaceutically acceptable salt thereof, a stereoisomer thereof, a tautomer thereof, a prodrug thereof, a solvate thereof or a hydrate thereof, and a drug for treating the diseases related to the expression of 5-HT3 receptor in the same or different specification unit preparation, together with pharmaceutically acceptable carriers, that were used for simultaneous or separate administration.

Further, said combined drug is a drug for the treatment of schizophrenia, and the drug for treating the diseases related to the expression of 5-HT3 receptor is selected from one or more of valproate, levometpromazine, alprazolam, droperidol, chlorpromazine, lipperidone, papperidone, olanzapine, ziprasidone, quetiapine, clozapine, lithium carbonate, diazepam, carbamazepine, selective serotonin reuptake inhibitors and tricyclic antidepressants;

or, the combined drug is a drug for the treatment of Parkinson's disease, and said drug for treating the diseases related to the expression of 5-HT3 receptor is selected from one or more of rotigotine, rezagilan, saffenamide, levodopa, carbidopa, dopamine agonist, COMT inhibitor, MAO-B inhibitor, amantadine and anticholinergic drugs;
or, the combined drug is a drug for the treatment of irritable bowel syndrome, and said drug for treating diseases related to the expression of 5-HT3 receptor is selected from the second serotonin 5-HT3 receptor regulator or serotonin 5-HT4 receptor regulator, wherein the second serotonin 5-HT3 receptor regulator or serotonin 5-HT4 receptor regulator is selected from one or more of alosetron, renzapril, cilansetron, tegaserod, prucalopride, ondansetron, somatostatin analogues, muscarinic receptor antagonists, laxatives, antispasmodics, antidepressants, antidiarrheal agents, prokinetic agents and peripheral opioid anesthetic antagonists;
or, the combined drug is a drug for the treatment of nausea or vomiting, and the drug for treating the diseases related to the expression of 5-HT3 receptor is selected from one or more of dexamethasone, alosetron, alprazolam, aripidem, dimenhydrinate, diphenhydramine, dorasetron, tetrahydrocannabinol, nabilone, dronabinol, daperidol, granisetron, droperidol, lorazepam, metoclopramide, midazolam, olanzapine, ondansetron, palonosetron, prochlorazine, promethazine and tropisetron.

In the present invention, the group -PMB is p-methoxybenzyl.

In the present invention, “substitution” means that a hydrogen in a molecule is substituted with other different atoms or groups, including the replacement of one, two or more hydrogens on the same or different atoms in the molecule.

C_{a-b} denotes all groups or molecules containing a-b carbon atoms, such as “C₁₋₈ alkyl” includes all branched or linear alkyls having 1-8 carbon atoms.

Experiments have proven that the compound of the preparation example has a strong affinity to 5-HT₃ receptors, and has high activity as a modulator of 5-HT₃ receptors; the present compound may be used to prepare a drug for treating irritable bowel syndrome, nausea, vomiting, gastroenteritis, gastric dysfunction, diarrhea, pain, carcinoid syndrome, drug addiction and other diseases; and the application prospects are broad.

Obviously, based on the above content of the present invention, according to the common technical knowledge and the conventional means in the field, without department from the above basic technical spirits, other various modifications, alternations, or changes can further be made.

By following specific examples of said embodiments, the above content of the present invention is further illustrated. But it should not be construed that the scope of the above subject matter of the present invention is limited to the following examples. The techniques realized based on the above content of the present invention are all within the scope of the present invention.

### EXAMPLES

The starting materials and equipment used in the present invention are all known, and could be obtained by purchasing commercially available articles.

### Example 1. Preparation of (S)-3-bromo-7-(quinuclidin-3-yl)-2,7,8,9-tetrahydro-6H-azepino[5,4,3-cd]indazole-6-one (compound 10) of the Present Invention

Compound 10 was prepared according to the following synthetic route:

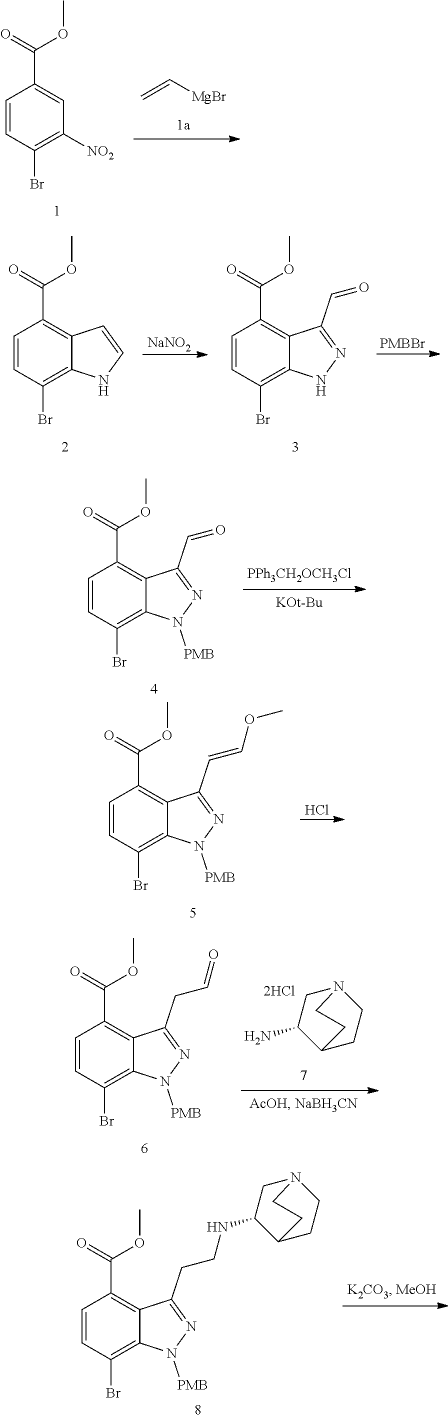

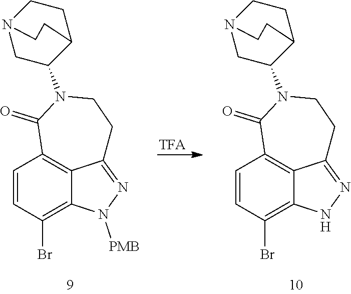

### Step 1. Preparation of methyl 7-bromo-1H-indole-4-carboxylate (compound 2)

To a solution of compound 1 (10.0 g, 38.5 mmol) in tetrahydrofuran (100 ml), was drop added compound 1a (115 mL, 115 mmol, 1 M tetrahydrofuran solution) under the protection of nitrogen at −40° C. After addition, the reaction solution was allowed to react at −40° C. for 1 h. The reaction solution was poured into the saturated aqueous solution of ammonium chloride to quench the reaction, and the resultant mixture was extracted twice with ethyl acetate. Ethyl acetate layer was combined, washed with saturated brine, dried over anhydrous sodium sulfate, and rotatory evaporated to dry. The residue was triturated in n-hexane to obtain pure compound 2 (3.9 g) as a white solid, with a yield of 40%.

Structural identification of the prepared compound: ¹HNMR (400 MHz, DMSO-d₆) δ 11.73 (s, 1H), 7.65-7.67 (d, 1H), 7.60-7.62 (dd, 1H), 7.07-7.08 (d, 1H), 3.90 (s, 3H). MS (ESI) m z: 254.2 [M+1]⁺.

### Step 2. Preparation of methyl 7-bromo-3-formyl-1H-indole-4-carboxylate (compound 3)

Compound 2 (3.90 g, 15.3 mmol) was added to a solution of sodium nitrite (12.7 g, 184 mmol) in water (200 mL), and the reaction solution was cooled to −5° C. in an ice-water bath. HCl aqueous solution (30.7 mL, 184 mmol, 6 M) was slowly drop added to the reaction solution. After addition, the reaction solution was allowed to react at −5° C. for 1 h, and then warmed to room temperature and stirred overnight. The reaction solution was extracted twice with ethyl acetate. Ethyl acetate layer was combined, washed with saturated brine, dried over anhydrous sodium sulfate, and rotatory evaporated to dry. The residue was triturated in petroleum ether/ethyl acetate to obtain pure compound 3 (1.84 g) as a pale yellow solid, with a yield of 42%.

Structural identification of the prepared compound: MS (ESI) m z: 283.0 [M+1]⁺

### Step 3. Preparation of methyl 7-bromo-3-formyl-1-(4-methoxybenzyl)-1H-indole-4-carboxylate (compound 4)

To a solution of compound 3 (1.84 g, 6.50 mmol) in DMF (20 ml), were added p-methoxybenzyl bromide (1.44 g, 7.15 mmol) and cesium carbonate (2.54 g, 7.80 mmol). The reaction solution was stirred at room temperature overnight. The reaction solution was poured into water and extracted twice with ethyl acetate. Ethyl acetate layer was combined, washed with water and saturated brine, dried over anhydrous sodium sulfate, and rotatory evaporated to dry. The residue was purified by silica gel column to obtain pure compound 4 (2.12 g) as a pale yellow solid, with a yield of 81%.

Structural identification of the prepared compound: ¹HNMR (400 MHz, CDCl₃) δ 10.86 (s, 1H), 7.95-7.97 (d, 1H), 7.66-7.68 (d, 1H), 9.39-7.44 (m, 2H), 6.83-6.86 (d, 2H), 6.11 (s, 2H), 3.98 (s, 3H), 3.78 (s, 3H).

### Step 4. Preparation of methyl 7-bromo-1-(4-methoxybenzyl)-3-(2-methoxyvinyl)-1H-indole-4-carboxylate (compound 5)

To a suspension of compound 4 (2.12 g, 5.26 mmol) and (methoxymethyl)triphenylphosphonium chloride (1.98 g, 5.78 mmol) in tetrahydrofuran (20 ml), was added potassium t-butoxide (708 mg, 6.31 mmol) in batches at 0° C. After addition, the reaction solution was warmed to room temperature and stirred for 1 h. The reaction solution was poured into water and extracted twice with ethyl acetate. Ethyl acetate layer was combined, washed with water and saturated brine, dried over anhydrous sodium sulfate, and rotatory evaporated to dry, to provide crude compound 5, which was directly used in the next step without further purification.

Structural identification of the prepared compound: MS (ESI) m z: 431.2 [M+1]⁺

### Step 5. Preparation of methyl 7-bromo-1-(4-methoxybenzyl)-3-(2-oxoethyl)-1H-indole-4-carboxylate (compound 6)

To a solution of compound 5 (crude, 5.26 mmol in theoretical amount) in tetrahydrofuran (20 ml), was added HCl aqueous solution (4.4 ml, 26.3 mmol, 6 M). The reaction solution was heated to 60° C. and allowed to react for 2 h. The reaction solution was poured into water and extracted twice with ethyl acetate. Ethyl acetate layer was combined, washed with saturated brine, dried over anhydrous sodium sulfate, and rotatory evaporated to dry. The residue was triturated in methyl t-butyl ether, to obtain pure compound 6 (1.03 g) as a pale yellow solid, with a two-step yield of 47%.

Structural identification of the prepared compound: ¹HNMR (400 MHz, CDCl₃) δ 9.57 (s, 1H), 7.74-7.76 (d, 1H), 7.58-7.60 (d, 1H), 7.09-7.11 (m, 2H), 6.84-6.86 (d, 2H), 5.69 (s, 2H), 4.38 (s, 3H), 3.89 (s, 3H), 3.79 (s, 3H).

### Step 6. Preparation of methyl 7-bromo-1-(4-methoxybenzyl)-3-(2-(quinuclidin-3-yl)ethyl)-1H-indole-4-carboxylate (compound 8)

To a solution of compound 6 (1.03 g, 2.47 mmol) in dichloromethane (20 mL), were added compound 7 (737 mg, 3.70 mmol) and glacial acetic acid (741 mg, 12.35 mmol). The reaction solution was stirred at room temperature for 6 h, to which was then added sodium cyanoborohydride (466 mg, 7.41 mmol), and the resultant solution was stirred overnight at room temperature. The reaction solution was poured into a saturated aqueous solution of sodium bicarbonate and extracted twice with dichloromethane. Dichloromethane layer was combined, dried over anhydrous sodium sulfate, and rotatory evaporated to dry. The residue was purified by silica gel column to provide pure compound 8 as a pale yellow solid (488 mg), with a yield of 37%.

Structural identification of the prepared compound: MS (ESI) m z: 527.2 [M+1]⁺

### Step 7. Preparation of (S)-3-bromo-2(4-methoxybenzyl)-7-(quinuclidin-3-yl)-2,7,8,9-tetrahydro-6H-azepino[5,4,3-cd]indazole-6-one (compound 9)

To a solution of compound 8 (488 mg, 0.925 mmol) in methanol (10 ml), was added potassium carbonate (256 mg, 1.85 mmol). The reaction solution was heated to 60° C. and allowed to react for 8 h. The reaction solution was taken out and cooled, poured into water, and extracted twice with ethyl acetate. Ethyl acetate layer was combined, washed with water and saturated brine, dried over anhydrous sodium sulfate, and rotatory evaporated to dry. The residue was purified by silica gel column to provide pure compound 9 as a pale yellow solid (372 mg), with a yield of 81%.

Structural identification of the prepared compound: MS (ESI) m z: 495.2 [M+1]⁺

### Step 8. Preparation of (S)-3-bromo-7-(quinuclidin-3-yl)-2,7,8,9-tetrahydro-6H-azepino[5,4,3-cd]indazole-6-one (compound 10)

To a suspension of compound 9 (100 mg, 0.202 mmol) in anisole (0.5 mL), was added trifluoroacetic acid (0.5 mL). The reaction solution was heated to 80° C. and allowed to react for 18 h. The reaction solution was taken out, cooled, and rotatory evaporated to dry. To the residue, was added HCl aqueous solution (5 mL), and then the resultant solution was washed twice with methyl t-butyl ether. The water layer was adjusted to pH 12 with a concentrated NaOH aqueous solution. The obtained suspension was stirred for 1 h, filtered, and washed with water. The filter cake was collected and dried to obtain pure compound 10 (62 mg) as a light yellow solid, with a yield of 82%.

Structural identification of the prepared compound: ¹HNMR (400 MHz, DMSO-d₆) δ 13.48 (br, 1H), 7.66-7.77 (m, 2H), 4.49 (br, 1H), 4.11 (br, 1H), 3.77 (br, 1H), 3.15-3.16 (m, 2H), 2.90-3.04 (m, 3H), 2.55-2.77 (m, 3H), 1.99 (s, 1H), 1.43-1.68 (m, 3H), 1.43 (m, 1H). MS (ESI) m z: 375.2 [M+1]⁺

### Example 2. Preparation of (S)-3-cyano-7-(quinuclidin-3-yl)-2,7,8,9-tetrahydro-6H-azepino[5,4,3-cd]indazole-6-one (compound 11) of the Present Invention

Compound 11 was prepared according to the following synthetic route:

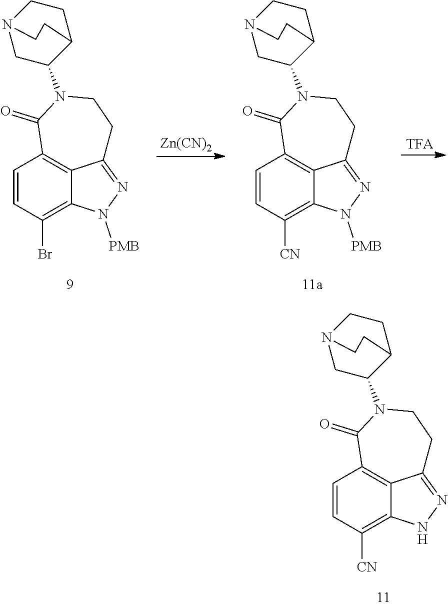

### Step 1. Preparation of (S)-3-cyano-2-(4-methoxybenzyl)-7-(quinuclidin-3-yl)-2,7,8,9-tetrahydro-6H-azepino[5,4,3-cd]indazole-6-one (compound 1a)

To a solution of compound 9 (100 mg, 0.202 mmol) in DMF (2 mL), were added zinc cyanide (47 mg, 0.40 mmol) and tetrakis(triphenylphosphine)palladium (30 mg), and then the reaction solution was purged with nitrogen, heated to 100° C., and allowed to react overnight. The reaction solution was taken out, cooled, poured into water, and extracted twice with ethyl acetate. Ethyl acetate layer was combined, washed with water and saturated brine, dried over anhydrous sodium sulfate, and rotatory evaporated to dry. The residue was purified by silica gel column to provide pure compound 11a as a pale yellow solid (72 mg), with a yield of 81%.

Structural identification of the prepared compound: MS (ESI) m z: 442.2 [M+1]⁺

### Step 2. Preparation of (S)-3-bromo-7-(quinuclidin-3-yl)-2,7,8,9-tetrahydro-6H-azepino[5,4,3-cd]indazole-6-one (compound 11)

To a suspension of compound 11a (72 mg, 0.163 mmol) in anisole (0.5 mL), was added trifluoroacetic acid (0.5 mL). The reaction solution was heated to 80° C. and allowed to react for 18 h. The reaction solution was taken out, cooled, and rotatory evaporated to dry. To the residue, was added HCl aqueous solution (5 mL), and then the resultant solution was washed twice with methyl t-butyl ether. The water layer was adjusted to pH 12 with a concentrated NaOH aqueous solution. The obtained suspension was stirred for 1 h, filtered, and washed with water. The filter cake was collected and dried to obtain pure compound 11 (40 mg) as a light yellow solid, with a yield of 76%.

Structural identification of the prepared compound: ¹HNMR (400 MHz, DMSO-d₆) δ 13.99 (br, 1H), 8.01-8.37 (d, 1H), 7.81-7.83 (d, 1H), 4.49 (br, 1H), 4.13 (br, 1H), 3.79 (br, 1H), 3.15-3.16 (m, 2H), 2.90-3.04 (m, 3H), 2.55-2.77 (m, 3H), 1.99 (s, 1H), 1.43-1.68 (m, 3H), 1.43 (m, 1H). MS (ESI) m z: 322.2 [M+1]⁺

### Example 3. Preparation of (S)-3-hydroxyl-7-(quinuclidin-3-yl)-2,7,8,9-tetrahydro-6H-azepino[5,4,3-cd]indazole-6-one (compound 12) of the Present Invention

Compound 12 was prepared according to the following synthetic route:

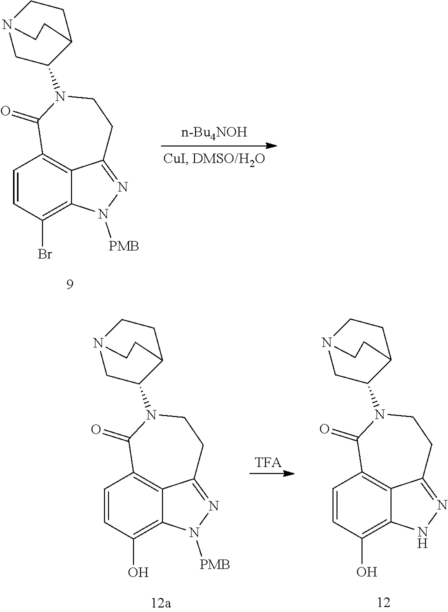

### Step 1. Preparation of (S)-3-hydroxyl-2-(4-methoxybenzyl)-7-(quinuclidin-3-yl)-2,7,8,9-tetrahydro-6H-azepino[5,4,3-cd]indazole-6-one (compound 12a)

To a solution of compound 9 (100 mg, 0.202 mmol) in water (1.5 mL) and DMSO (1 mL), were added tetrabutylammonium hydroxide (157 mg, 0.606 mmol), 8-hydroxyquinaldine (13 mg, 0.082 mmol) and CuI (8 mg, 0.042 mmol), and then the reaction solution was purged with nitrogen. The reaction solution was heated to 100° C., and allowed to react overnight. The reaction solution was taken out, cooled, and poured into water, followed by extraction with ethyl acetate. Ethyl acetate layer was combined, washed with water and saturated brine, dried over anhydrous sodium sulfate, and rotatory evaporated to dry. The residue was purified by silica gel column to provide pure compound 12a (53 mg) as a pale yellow solid, with a yield of 61%.

Structural identification of the prepared compound: MS (ESI) m z: 451.2 [M+18]⁺

### Step 2. Preparation of (S)-3-hydroxyl-7-(quinuclidin-3-yl)-2,7,8,9-tetrahydro-6H-azepino[5,4,3-cd]indazole-6-one (compound 12)

To a suspension of compound 12a (53 mg, 0.123 mmol) in anisole (0.5 mL), was added trifluoroacetic acid (0.5 mL). The reaction solution was heated to 80° C. and allowed to react for 18 h. The reaction solution was taken out, cooled, and rotatory evaporated to dry. To the residue, was added HCl aqueous solution (5 mL), and then the resultant solution was washed twice with methyl t-butyl ether. The water layer was adjusted to pH 12 with a concentrated NaOH aqueous solution. The obtained suspension was stirred for 1 h, filtered, and washed with water. The filter cake was collected and dried to obtain pure compound 12 (22 mg) as a pale yellow solid, with a yield of 57%.

Structural identification of the prepared compound: MS (ESI) m z: 313.2 [M+1]⁺

### Example 4. Preparation of (S)-3-deutero-7-(quinuclidin-3-yl)-2,7,8,9-tetrahydro-6H-azepino[5,4,3-cd]indazole-6-one (compound 13) of the present invention

Compound 13 was prepared according to the following synthetic route:

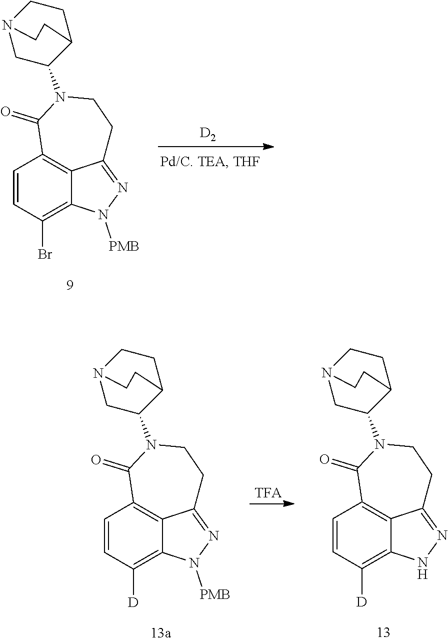

### Step 1. Preparation of (S)-3-deutero-2-(4-methoxybenzyl)-7-(quinuclidin-3-yl)-2,7,8,9-tetrahydro-6H-azepino[5,4,3-cd]indazole-6-one (compound 13a)

To a solution of compound 9 (100 mg, 0.202 mmol) in tetrahydrofuran (5 ml), was added triethylamine (61 mg, 0.606 mmol), and then the reaction solution was purged with deuterium gas, and stirred overnight under deuterium gas (balloon). The reaction solution was filtered, and washed with tetrahydrofuran. The filtrate was rotatory evaporated to dry. The residue was purified by silica gel column to provide pure compound 13a (70 mg) as a pale yellow solid, with a yield of 83%.

Structural identification of the prepared compound: MS (ESI) m z: 418.2 [M+1]⁺

### Step 2. Preparation of (S)-3-deutero-7-(quinuclidin-3-yl)-2,7,8,9-tetrahydro-6H-azepino[5,4,3-cd]indazole-6-one (compound 13)

To a suspension of compound 13a (70 mg, 0.168 mmol) in anisole (0.5 mL), was added trifluoroacetic acid (0.5 mL). The reaction solution was heated to 80° C. and allowed to react for 18 h. The reaction solution was taken out, cooled, and rotatory evaporated to dry. To the residue, was added HCl aqueous solution (5 mL), and then the resultant solution was washed twice with methyl t-butyl ether. The water layer was adjusted to pH 12 with a concentrated NaOH aqueous solution. The obtained suspension was stirred for 1 h, filtered, and washed with water. The filter cake was collected and dried to obtain pure compound 13 (42 mg) as a pale yellow solid, with a yield of 84%.

Structural identification of the prepared compound: MS (ESI) m z: 298.2 [M+1]⁺

### Example 5. Preparation of (S)-3-fluoro-7-(quinuclidin-3-yl)-2,7,8,9-tetrahydro-6H-azepino[5,4,3-cd]indazole-6-one (compound 14) of the present invention

Compound 14 was prepared according to the following synthetic route:

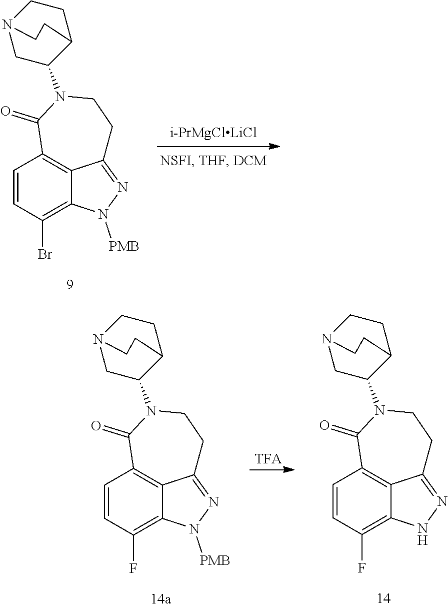

### Step 1. Preparation of (S)-3-fluoro-2-(4-methoxybenzyl)-7-(quinuclidin-3-yl)-2,7,8,9-tetrahydro-6H-azepino[5,4,3-cd]indazole-6-one (compound 14a)

To a solution of compound 9 (100 mg, 0.202 mmol) in tetrahydrofuran (5 ml), was added isopropylmagnesium chloride lithium chloride complex (0.23 mL, 0.30 mmol, 1.3 M tetrahydrofuran solution) dropwise at 0° C. After addition, the reaction solution was stirred at 0° C. for 1 h. The reaction solution was warmed to room temperature and concentrated to dry under reduced pressure. To the residue, was added dichloromethane (1 mL), and then the system was purged with nitrogen. The reaction solution was cooled to −70° C., to which was drop added the solution of n-fluorobenzenesulfonimide (95 mg, 0.30 mmol) in perfluorodecalin (0.5 mL) and dichloromethane (1 mL). After addition, the reaction solution was stirred at 0° C. for 30 min, and then stirred at room temperature for 2 h. The reaction solution was poured to a saturated aqueous solution of ammonium chloride to quench the reaction, and the resultant solution was extracted twice with dichloromethane. Dichloromethane layer was combined, dried over anhydrous sodium sulfate, and rotatory evaporated to dry. The residue was purified by silica gel column to provide pure compound 14a (45 mg) as a pale yellow solid, with a yield of 51%.

Structural identification of the prepared compound: MS (ESI) m z: 435.2 [M+1]⁺

### Step 2. Preparation of (S)-3-fluoro-7-(quinuclidin-3-yl)-2,7,8,9-tetrahydro-6H-azepino[5,4,3-cd]indazole-6-one (compound 14)

To a suspension of compound 14a (45 mg, 0.104 mmol) in anisole (0.5 mL), was added trifluoroacetic acid (0.5 mL). The reaction solution was heated to 80° C. and allowed to react for 18 h. The reaction solution was taken out, cooled, and rotatory evaporated to dry. To the residue, was added HCl aqueous solution (4 mL), and then the resultant solution was washed twice with methyl t-butyl ether. The water layer was adjusted to pH 12 with a concentrated NaOH aqueous solution. The obtained suspension was stirred for 1 h, filtered, and washed with water. The filter cake was collected and dried to obtain pure compound 14 (26 mg) as a pale yellow solid, with a yield of 80%.

Structural identification of the prepared compound: MS (ESI) m z: 315.4 [M+1]⁺

### Example 6. Preparation of (S)-3-chloro-7-(quinuclidin-3-yl)-2,7,8,9-tetrahydro-6H-azepino[5,4,3-cd]indazole-6-one (compound 15)

Compound 15 was prepared according to the following synthetic route:

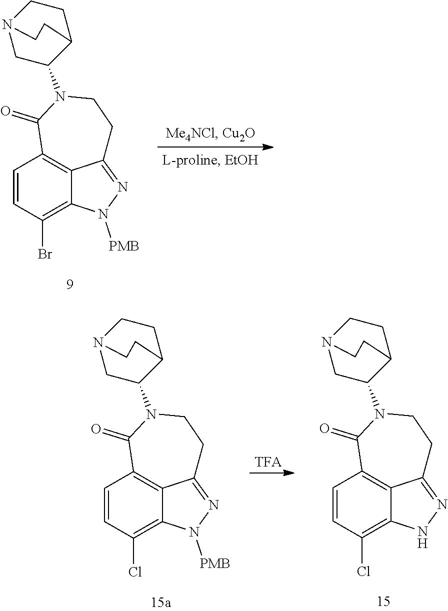

### Step 1. Preparation of (S)-3-chloro-2-(4-methoxybenzyl)-7-(quinuclidin-3-yl)-2,7,8,9-tetrahydro-6H-azepino[5,4,3-cd]indazole-6-one (compound 15a)

Compound 9 (100 mg, 0.202 mmol), ethanol (1.5 mL), tetramethylammonium chloride (66 mg, 0.606 mmol), L-proline (9.2 mg, 0.08 mmol) and CuO (5.7 mg, 0.04 mmol) were added to the sealed tube. The reaction solution was purged with nitrogen, sealed, heated to 110° C., and stirred for 20 h. The reaction solution was cooled, poured into the aqueous solution and extracted twice with ethyl acetate. Ethyl acetate layer was combined, dried over anhydrous sodium sulfate, and rotatory evaporated to dry. The residue was purified by silica gel column to provide pure compound 15a (81 mg) as a pale yellow solid, with a yield of 89%.

Structural identification of the prepared compound: MS (ESI) m z: 451.3 [M+1]⁺

### Step 2. Preparation of (S)-3-chloro-7-(quinuclidin-3-yl)-2,7,8,9-tetrahydro-6H-azepino[5,4,3-cd]indazole-6-one (compound 15)

To a suspension of compound 15a (81 mg, 0.18 mmol) in anisole (0.5 mL), was added trifluoroacetic acid (0.5 mL). The reaction solution was heated to 80° C. and allowed to react for 18 h. The reaction solution was taken out, cooled, and rotatory evaporated to dry. To the residue, was added HCl aqueous solution (5 mL), and then the resultant solution was washed twice with methyl t-butyl ether. The water layer was adjusted to pH 12 with a concentrated NaOH aqueous solution. The obtained suspension was stirred for 1 h, filtered, and washed with water. The filter cake was collected and dried to obtain pure compound 15 (52 mg) as a pale yellow solid, with a yield of 87%.

Structural identification of the prepared compound: ¹HNMR (400 MHz, DMSO-d₆) δ 13.57 (br, 1H), 7.46-7.66 (d, 1H), 7.57-7.59 (d, 1H), 4.49 (br, 1H), 4.13 (br, 1H), 3.79 (br, 1H), 3.15-3.16 (m, 2H), 2.90-3.04 (m, 3H), 2.55-2.77 (m, 3H), 1.99 (s, 1H), 1.43-1.68 (m, 3H), 1.43 (m, 1H).

MS (ESI) m/z: 331.2 [M+1]⁺

In the following, the beneficial effect of the present invention was demonstrated by the experimental example.

### Experimental Example 1. Evaluation on the Affinity of the Compound According to the Present Invention for Human 5-HT₃ Receptor

### 1. Experimental Method:

The relative affinity of the compound to human 5-HT3 receptor was detected by scintillation proximity assay (SPA). The specific procedures were as follows: the test compound was diluted to 10 mM with 100% DMSO, and then subjected to 10× gradient dilution with 100% DMSO in a 96-well plate, and each of the resultant solution was further diluted with the test buffer to provide 4× test concentration. The sample, 10 nM of [9-methyl-3H]BRL-43694, 3 μg of human 5-HT3 receptor membrane, and 0.5 mg/mL of SPA beads were incubated in 50 mM Tris-HCl (pH 7.5, 3 mM MgCl₂, 1 mM EDTA and 10% DMSO), with a final volume of 0.2 mL. By successively adding 50 μL of competitive test compound or buffer, SPA beads, radioactive ligands and 5-HT3 receptor membrane, the binding reaction was performed in the wells of PicoPlates-96 well plate. After it was stirred and incubated overnight at room temperature, the plate was centrifuged at 1500 rpm for 15 min, and then incubated in dark for 30 min. Finally, the 5-min radioactivity was read in the TopCount microplate counter. Total binding control only contained the above buffer used to dilute the test compound, while the nonspecific binding was determined at the presence of 30 μM MDL-72222, and thus the specific binding was deduced by the total binding control minus the nonspecific binding. All experiments were carried out by ten concentrations of competitive ligand, and each group was established multiple holes. ALB-137391 was used as positive control.

ALB-137391 was a 5-HT3 receptor partial agonist, and provided by AMRI company, USA, with a production batch number of 1631-B-R0-01-43-01. The structure was as follows:

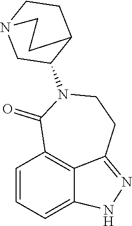 [SMILES: O=C1c2cccc3[nH]nc(c23)CCN1[C@@H]1CN2CCC1CC2]

### 2. Experimental Data and Analysis:

The half inhibitory concentration (IC₅₀) was determined from the specific binding data by using XLfit4.1 curve fitting software of IDBS Ltd.

Inhibition constant Ki=IC₅₀/(1+(L/KD)), wherein L=the concentration of radioactive ligands in the test, KD=the affinity of radioactive ligands to receptors.

### 3. Experimental Results:

The affinity and IC₅₀ values of the test compound of the present invention and the positive control ALB-137391 for human 5-HT3 receptor were shown in the Table below.

TABLE 1
Test results for the affinity of each compound to
human 5-HT₃ receptor.

| Compounds | IC₅₀ (nmol) | Ki (nmol) |
|---|---|---|
| 10 | 34.24 | 6.20 |
| 11 | 359.4 | 65.11 |
| 12 | 13.99 | 2.53 |
| 13 | 3.315 | 0.60 |
| 14 | 73.98 | 13.40 |
| 15 | 47.06 | 8.53 |
| ALB-137391 | 4.478 | 0.81 |

As shown in Table 1, the compound of the present invention has a strong affinity to human 5-HT3 receptor (especially compounds 10, 12, 13, 14 and 15), and has a high activity as a 5-HT3 receptor regulator. In addition, the affinity and inhibitory effect of compound 13 of the present invention on 5-HT3 receptor were even better than that of positive control ALB-137391.

In summary, the present invention provided compound of formula I and its preparation method. Compound of formula I could be used as a 5-HT3 receptor regulator. Experiments had proven that the compound of the preparation example had a strong affinity to 5-HT3 receptors, and has a high activity as a 5-HT3 receptor modulator. The present compound could be used to prepare a drug for the treatment of irritable bowel syndrome, nausea, vomiting, gastroenteritis, gastric dysfunction, diarrhea, pain, carcinoid syndrome, drug addiction and other diseases, and thus had a broad application prospect.

1. Compound of formula I, a pharmaceutically acceptable salt thereof, a stereoisomer thereof, a tautomer thereof, a prodrug thereof, a solvate thereof or a hydrate thereof: 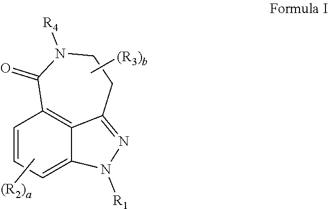
wherein, each of R₁, R₂, R₃, and R₄ is independently selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR₁₀, —SO₂R₁, -L₂-CN, substituted or unsubstituted alkenyl, substituted or unsubstituted alkynyl, substituted or unsubstituted alkyl, substituted or unsubstituted alkoxy, substituted or unsubstituted monoheterocyclyl, substituted or unsubstituted monocycloalkyl, substituted or unsubstituted fused azacycloalkyl, substituted or unsubstituted bridged azacycloalkyl, substituted or unsubstituted azabi(cycloalkyl), substituted or unsubstituted azaspirocycloalkyl, substituted or unsubstituted fused cycloalkyl, substituted or unsubstituted bridged cycloalkyl, substituted or unsubstituted bi(cycloalkyl), and substituted or unsubstituted spirocycloalkyl;
above substitutents are each independently selected from the group consisting of deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR₁₀, —SO₂R¹¹, -L₂-CN, C₂₋₈ alkenyl, C₂₋₈ alkynyl, C₁₋₈ alkyl, and C₁₋₈ alkoxy;
wherein, each of L₀, L₁, L₂, L₃, L₄, L₅, and L₆ is independently selected from 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, and C₁₋₈ alkyl;
a is selected from an integer of 1 to 3, and b is selected from an integer of 1 to 4.

2. The compound according to claim 1, a pharmaceutically acceptable salt thereof, a stereoisomer thereof, a tautomer thereof, a prodrug thereof, a solvate thereof or a hydrate thereof, characterized in that:
R₂ is selected from the group consisting of deuterium, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, substituted or unsubstituted C₂₋₈ alkenyl, substituted or unsubstituted C₂₋₈ alkynyl, substituted or unsubstituted C₁₋₈ alkyl, substituted or unsubstituted C₁₋₈ alkoxy, substituted or unsubstituted 3-10 membered monoheterocyclyl, substituted or unsubstituted 3-10 membered monocycloalkyl, substituted or unsubstituted 3-10 membered fused azacycloalkyl, substituted or unsubstituted 3-10 membered bridged azacycloalkyl, substituted or unsubstituted 3-10 membered azabi(cycloalkyl), substituted or unsubstituted 3-10 membered azaspirocycloalkyl, substituted or unsubstituted 3-10 membered fused cycloalkyl, substituted or unsubstituted 3-10 membered bridged cycloalkyl, substituted or unsubstituted 3-10 membered bi(cycloalkyl), and substituted or unsubstituted 3-10 membered spirocycloalkyl;
above substituents, L₀, L₁, L₂, L₃, L₄, L₅, L₆, R⁶, R⁷, R⁸, R⁹, R¹⁰, and R¹¹ are as described in claim 1;
R₁, R₃, R₄, a, and b are as described in claim 1;

3. The compound according to claim 1, a pharmaceutically acceptable salt thereof, a stereoisomer thereof, a tautomer thereof, a prodrug thereof, a solvate thereof or a hydrate thereof, characterized in that:
said compound has a structure of formula II-1a: 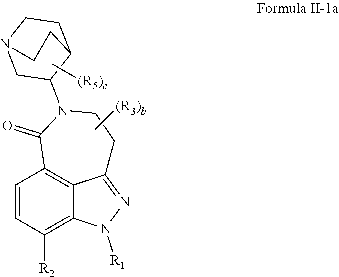
wherein, R₂ is selected from the group consisting of deuterium, cyano or hydroxyl;
R₅ is selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, C₂₋₈ alkenyl, C₂₋₈ alkynyl, C₁₋₈ alkyl, C₁₋₈ alkoxy; wherein, each of L₀, L₁, L₂, L₃, L₄, L₅, and L₆ is independently selected from the group consisting of 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, C₁₋₈ alkyl;
c is selected from an integer of 1 to 5;
R₁, R₃, and b are as described in claim 1.

4. The compound according to claim 3, a pharmaceutically acceptable salt thereof, a stereoisomer thereof, a tautomer thereof, a prodrug thereof, a solvate thereof or a hydrate thereof, characterized in that:
Said compound has a structure of formula II-2a: 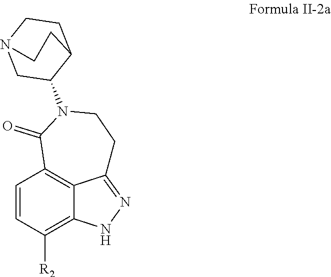
wherein, R₂ is selected from the group consisting of deuterium, cyano or hydroxyl.

5. The compound according to claim 1, a pharmaceutically acceptable salt thereof, a stereoisomer thereof, a tautomer thereof, a prodrug thereof, a solvate thereof or a hydrate thereof, characterized in that:
R₂ is selected from the group consisting of hydrogen or halogen;
R₁ is selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR₁₀, —SO₂R¹¹, -L₂-CN, substituted or unsubstituted C₂₋₈ alkenyl, substituted or unsubstituted C₂₋₈ alkynyl, substituted or unsubstituted C₁₋₈ alkoxy, substituted or unsubstituted 3-10 membered saturated monoheterocyclyl, substituted or unsubstituted 3-10 membered saturated monocycloalkyl, substituted or unsubstituted 3-10 membered saturated fused azacycloalkyl, substituted or unsubstituted 3-10 membered saturated bridged azacycloalkyl, substituted or unsubstituted 3-10 membered saturated azabi(cycloalkyl), substituted or unsubstituted 3-10 membered saturated azaspirocycloalkyl, substituted or unsubstituted 3-10 membered saturated fused cycloalkyl, substituted or unsubstituted 3-10 membered saturated bridged cycloalkyl, substituted or unsubstituted 3-10 membered saturated bi(cycloalkyl), and substituted or unsubstituted 3-10 membered saturated spirocycloalkyl;
each of above substituents is independently selected from the group consisting of deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR₁₀, —SO₂R¹¹, -L₂-CN, C₂₋₈ alkenyl, C₂₋₈ alkynyl, C₁₋₈ alkyl, and C₁₋₈ alkoxy;
wherein, each of L₀, L₁, L₂, L₃, L₄, L₅, and L₆ is independently selected from 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, and C₁₋₈ alkyl;
a, b, R₃, and R₄ are as described in claim 1.

6. The compound according to claim 5, a pharmaceutically acceptable salt thereof, a stereoisomer thereof, a tautomer thereof, a prodrug thereof, a solvate thereof or a hydrate thereof, characterized in that said compound has a structure of formula III-1: 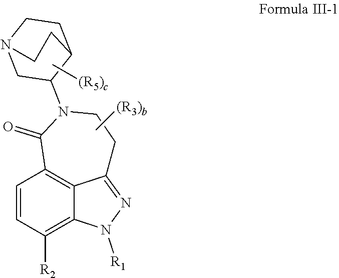
wherein, R₂ is selected from the group consisting of hydrogen or halogen;
R₅ is selected from the group consisting of hydrogen, deuterium, halogen, cyano, hydroxyl, carboxyl, nitro, amino, -L₀-OH, -L₃-C(O)R⁶, -L₄-CO(O)R⁷, -L₅-(O)COR⁸, -L₆-NHC(O)R⁹, -L₁-C(O)NHR¹⁰, —SO₂R¹¹, -L₂-CN, C₂₋₈ alkenyl, C₂₋₈ alkynyl, C₁₋₈ alkyl, C₁₋₈ alkoxy; wherein, each of L₀, L₁, L₂, L₃, L₄, L₅, and L₆ is independently selected from 0-8 alkylenes; each of R⁶, R⁷, R⁸, R⁹, R¹⁰, and R¹¹ is independently selected from the group consisting of hydrogen, deuterium, halogen, cyano, hydroxyl, carboxyl, nitro, amino, C₁₋₈ alkyl;
c is selected from an integer of 1 to 5;
R₁, R₃, and b are as described in claim 5.

7. The compound according to claim 6, a pharmaceutically acceptable salt thereof, a stereoisomer thereof, a tautomer thereof, a prodrug thereof, a solvate thereof or a hydrate thereof, characterized in that said compound has a structure of formula III-2: 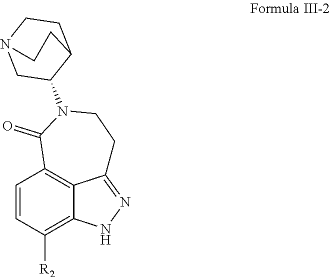
Wherein, R₂ is selected from halogen, and preferably fluorine, chlorine or bromine.

8. The compound according to claim 1, a pharmaceutically acceptable salt thereof, a stereoisomer thereof, a tautomer thereof, a prodrug thereof, a solvate thereof or a hydrate thereof, characterized in that said pharmaceutically acceptable salts include hydrochloride, hydrobromate, sulfate, phosphate, methanesulfonate, trifluoromethanesulfonate, benzenesulfonate, p-toluenesulfonate, 1-naphthalenesulfonate, 2-naphthalenesulfonate, acetate, trifluoroacetate, malate, tartrate, citrate, lactate, oxalate, succinate, fumarate, maleate, benzoate salicylate, phenylacetate, and mandelate.

9. A preparation method of the compound according to claim 4, characterized in that compound 9a has a deprotection reaction with a deprotection agent, to remove the group -PMB and obtain the final compound; the structure of compound 9a is 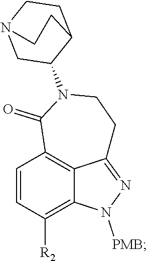
or, compound 9 is first subjected to a substitution reaction with a substitution reagent, to substitute Br in compound 9 with R₂ (other than Br) and obtain an intermediate product; then, the intermediate product has a deprotection reaction with the deprotection agent to remove the group -PMB and obtain the final compound; the structure of compound 9 is 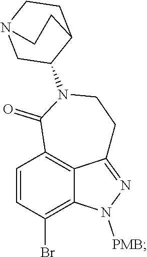
said final compound is the compound of claim 4;
said R₂ is selected from the group consisting of deuterium, cyano, hydroxyl or halogen, and said halogen is preferably fluorine, chlorine or bromine;
said deprotection agent is preferably trifluoroacetic acid; said substitution reagent is preferably zinc cyanide, 8-hydroxyquinaldine, deuterium gas, N-fluorobenzenesulfonimide or tetramethylammonium chloride.

10. The method according to claim 9, characterized in that the preparation method of compound 9a comprises the following steps:
(1) Compound 1b reacts with compound 1a, to obtain compound 2b;
(2) Compound 2b reacts with sodium nitrite, to obtain compound 3b;
(3) Compound 3b reacts with p-methoxybenzyl bromide, to obtain compound 4b;
(4) Compound 4b reacts with (methoxymethyl)triphenylphosphonium chloride, to obtain compound 5b;
(5) Compound 5b reacts with hydrochloric acid, to obtain compound 6b;
(6) Compound 6b reacts with compound 7, to obtain compound 8b;
(7) Compound 8b reacts with K₂CO₃, to obtain compound 9a;
wherein, the structure of compound 1b is 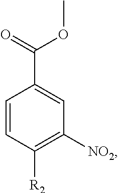
the structure of compound 1a is 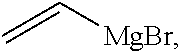
the structure of compound 2b is 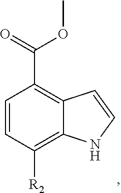
the structure of compound 3b is 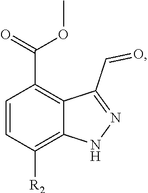
the structure of compound 4b is 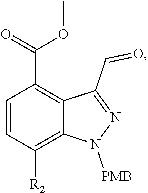
the structure of compound 5b is 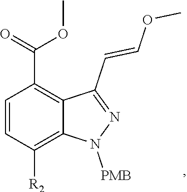
the structure of compound 6b is 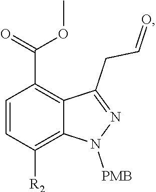
the structure of compound 7 is 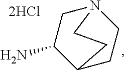
the structure of compound 8b is 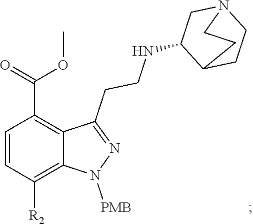
R₂ is as described in claim 9.

11. The use of the compound according to claim 1, a pharmaceutically acceptable salt thereof, a stereoisomer thereof, a tautomer thereof, a prodrug thereof, a solvate thereof or a hydrate thereof in the preparation of 5-HT3 receptor modulator.

12. The use according to claim 11, characterized in that said 5-HT3 receptor modulator is 5-HT3 receptor antagonist or 5-HT3 receptor partial agonist.

13. The use according to claim 11, characterized in that said 5-HT3 receptor modulator is a drug for treating diseases, and said diseases include generalized anxiety disorder, social phobia, vertigo, obsessive-compulsive disorder, panic disorder, post-traumatic stress disorder, bulimia nervosa, abstinence reaction, alcohol dependence, pain, sleep related central apnea, chronic fatigue syndrome, central nervous system related diseases, psychosis associated with Parkinson's disease, schizophrenia, cognitive impairment and deficits in schizophrenia, Parkinson's disease, Huntington's disease, presenile dementia, Alzheimer's disease, obesity, drug abuse disorders, dementia associated with neurodegenerative diseases, cognitive impairment, fibromyalgia syndrome, acne erythematosa, 5-hydroxytryptamine-mediated cardiovascular diseases, nausea, vomiting, gastrointestinal diseases, gastroesophageal reflux disease, Burkitt lymphoma, bronchial asthma, pruritus, migraine, epilepsy, carcinoid syndrome and irritable bowel syndrome; said nausea or vomiting is preferably that caused by chemotherapy, surgery, and radiotherapy.

14. A pharmaceutical composition, characterized in that it is a preparation obtained by using the compound according to claim 1, a pharmaceutically acceptable salt thereof, a stereoisomer thereof, a tautomer thereof, a prodrug thereof, a solvate thereof or a hydrate thereof as the active ingredient, with the addition of pharmaceutically acceptable excipients.

15. A combined drug, characterized in that it contains the compound according to claim 1, a pharmaceutically acceptable salt thereof, a stereoisomer thereof, a tautomer thereof, a prodrug thereof, a solvate thereof or a hydrate thereof, and a drug for treating the diseases related to the expression of 5-HT3 receptor in the same or different specification unit preparation, together with pharmaceutically acceptable carriers, that were used for simultaneous or separate administration.

16. The combined drug according to claim 15, characterized in that said combined drug is a drug for the treatment of schizophrenia, and the drug for treating the diseases related to the expression of 5-HT3 receptor is selected from one or more of valproate, levometpromazine, alprazolam, droperidol, chlorpromazine, lipperidone, papperidone, olanzapine, ziprasidone, quetiapine, clozapine, lithium carbonate, diazepam, carbamazepine, selective serotonin reuptake inhibitors and tricyclic antidepressants;
or, the combined drug is a drug for the treatment of Parkinson's disease, and said drug for treating the diseases related to the expression of 5-HT3 receptor is selected from one or more of rotigotine, rezagilan, saffenamide, levodopa, carbidopa, dopamine agonist, COMT inhibitor, MAO-B inhibitor, amantadine and anticholinergic drugs;
or, the combined drug is a drug for the treatment of irritable bowel syndrome, and said drug for treating diseases related to the expression of 5-HT3 receptor is selected from the second serotonin 5-HT3 receptor regulator or serotonin 5-HT4 receptor regulator, wherein the second serotonin 5-HT3 receptor regulator or serotonin 5-HT4 receptor regulator is selected from one or more of alosetron, renzapril, cilansetron, tegaserod, prucalopride, ondansetron, somatostatin analogues, muscarinic receptor antagonists, laxatives, antispasmodics, antidepressants, antidiarrheal agents, prokinetic agents and peripheral opioid anesthetic antagonists;
or, the combined drug is a drug for the treatment of nausea or vomiting, and the drug for treating the diseases related to the expression of 5-HT3 receptor is selected from one or more of dexamethasone, alosetron, alprazolam, aripidem, dimenhydrinate, diphenhydramine, dorasetron, tetrahydrocannabinol, nabilone, dronabinol, daperidol, granisetron, droperidol, lorazepam, metoclopramide, midazolam, olanzapine, ondansetron, palonosetron, prochlorazine, promethazine and tropisetron.
